(19) United States
(12) Patent Application Publication
(10) Pub. No.: US 2014/0128735 A1
(43) Pub. Date: May 8, 2014

(54) WIRELESS REAL-TIME ELECTROCARDIOGRAM AND MEDICAL IMAGE INTEGRATION

(71) Applicants: Todd Newell, Kent, WA (US); Eric Brinster, Everett, WA (US)

(72) Inventors: Todd Newell, Kent, WA (US); Eric Brinster, Everett, WA (US)

(73) Assignee: Cardiac Science Corporation, Waukesha, WI (US)

(21) Appl. No.: 13/836,755

(22) Filed: Mar. 15, 2013

Related U.S. Application Data
(60) Provisional application No. 61/721,655, filed on Nov. 2, 2012.

(51) Int. Cl. A61B 8/08; A61B 8/00; A61B 5/0456; A61B 5/00; A61B 5/04; A61B 8/14; A61B 5/044

(52) U.S. Cl. CPC A61B 8/5284; A61B 8/14; A61B 8/0883; A61B 8/56; A61B 8/463; A61B 5/044; A61B 5/0006; A61B 5/04012; A61B 5/0456
USPC 600/440; 600/443

(57) ABSTRACT

A medical data acquisition and display system utilizing separate data signals to transmit both high-resolution data and low-latency synchronization data acquired from patient sensors utilizing different wireless protocols. The low-latency stream of data transmitted over a radio frequency transmission as a timing-pulse in real-time. This high-resolution data includes detailed sensor readings from the patient and also includes digital markers (i.e., the timing-pulse) identifying the temporal location of the high-resolution data relative to imaging data that is simultaneously acquired from a patient. The high-resolution data can be electrocardiogram (ECG) data. The timing pulse can be based on the physiological QRS complex within the ECG data. The imaging data can be echocardiogram imagery of a heart that generated the ECG data. The echocardiogram imagery and the high-resolution ECG data are presented simultaneously by aligning the transmitted digital markers with the physiological QRS complex within the ECG data.

The present application claims the benefit of U.S. Provisional Application No. 61/721,655 entitled WIRELESS REAL-TIME ELECTROCARDIOGRAM AND MEDICAL IMAGE INTEGRATION and filed Nov. 2, 2012, which is incorporated herein in its entirety by reference.

### FIELD OF THE INVENTION

The inventions herein relate to the medical field of cardiovascular stress tests and generally to the area of remote diagnostic monitoring. More particularly, the present inventions generally relate to systems and methods of wireless collection, transmission, synchronization and presentation of cardiovascular stress electrocardiogram (ECG) and echocardiogram (echo) data over multiple channels.

### BACKGROUND OF THE INVENTION

A body surface electrocardiogram (ECG) is a measure of the electrical activity of a patient's heart. This electrical activity of the heart can be monitored with various medical devices including wireless medical devices. One example of such a monitor includes the wireless ECG CardioPart 12 Blue diagnostic product produced by Amedtec Medizintechnik Aue GmbH of Germany. Another example of wireless ECG monitoring technology is the system and method disclosed in U.S. Pat. No. 6,611,705 to Hopman et al., which is incorporated by reference herein. These devices monitor the electrical activity of the heart through a plurality of sensors or electrodes on the surface of a patient's body. For medical diagnostic purposes, a patient's ECG signals are often monitored during a cardiovascular exercise stress test or chemical stress test, conducted under the supervision of a medical professional, while the patient is engaged in various states of activity which can stress the patient's heart. The signals acquired in the stress test conducted are typically plotted as a varying intensity electrical wave over the period of time they were acquired from the patient.

Ultrasonic diagnostic imaging systems, such as echocardiogram (echo) or other ultrasound echo systems, can provide a medical professional with images of a patient's anatomy from various perspectives. Both internal and external ultrasound imaging devices are known. Additional imaging techniques such as magnetic resonance imaging (MRI) are also available to medical professionals to obtain cardiac images. One example of an ultrasound image acquisition technique is described in U.S. Pat. No. 6,488,629 to Saetre et al., which is incorporated by reference herein.

Some previous efforts have been made to integrate stress test ECG data with real-time ultrasound echo tests. Attempts to integrate these tests generally rely upon physical connections that provide electrical signals linking both echo and ECG data-acquisition devices with image display systems to display the echo and ECG information. Echo imaging systems typically require a very low data latency with a constant phase lag generally less than or equal to 35 milliseconds. Known products use a hardwired patient preamp module that has a cable tethered to the patient. Digital transmission of the QRS complex component of an ECG signal via hardwired TTL or analog channels is standard practice in the industry.

Existing integrations of stress tests and echo imaging are conducted with physical connections carrying an analog signal linking an ECG data-acquisition preamp attached to the patient with the echo display system to provide QRS synchronization information. This signal is typically an analog signal representing one of the ECG lead signals (e.g., lead I, II, III, V2 or V5) of a conventional ECG sensor array. Echo systems typically require that the QRS synchronization information have a very low data-latency with a constant phase lag, typically no more than 35 milliseconds, in order to synchronize the echo images with the QRS pulses.

There are also known devices related to wireless transmission of patient data and digital pulses that represent the QRS complex in heart monitoring applications. However, the low latency requirements of echo imaging systems makes the use of existing wireless technology protocols, such as Bluetooth and Wi-Fi, unsuitable for transmission of QRS synchronization information in real time. Accordingly, current designs are largely limited to use of a hardwired, physical cable-connection between an ECG data-acquisition preamp module and an echo display system.

### SUMMARY OF THE INVENTION

The present invention achieves both reliable wireless data transmission and low data-transmission latency by separating acquired data signals into multiple parts, and re-synchronizing the data with a host processor. Embodiments of the present invention combine low-latency real-time QRS data, in the form of a temporal marker, on one wireless channel, and high-bandwidth, high-resolution ECG data on a second wireless channel that may be of a higher-latency due to error correction and signal quality efforts. The two separate channels are transmitted wirelessly to a host system where the two data streams are then resynchronized to provide both low latency and reliable data delivery that can be combined with real-time medical images that were simultaneously acquired with the ECG data.

Existing ECG stress testing systems do not use wireless technology when integrating ECG data with medical imaging systems due to the demanding data-latency requirements of real-time medical imaging. Therefore, the present invention provides an advantageous solution to an existing problem in the field of medical diagnostics.

Embodiments of the present invention can provide a means to integrate data received from a patient during a stress test over a wireless connection with any other system or device requiring an ECG signal and a QRS trigger. Embodiments of the present invention can provide rich, diagnostic quality, twelve-lead ECG data for echo integration and analysis in real-time.

Embodiments of the present invention include a combination of high-reliability wireless protocols coupled with a real-time (i.e., low-latency) radio channel to transmit time sensitive synchronization data. Imaging data can be transmitted either wirelessly or through a cabled connection coupling an ultrasound transducer with an image display panel for synchronization with the two wireless channels.

Embodiments of the present invention separate the data signals from patient sensors into multiple parts, and transmit the embedded information over at least two streams of wireless transmission utilizing different wireless protocols. The first stream of data can be transmitted using a standardized radio frequency (RF) transmission, for example, a timing-pulse in real-time over a 915 MHz carrier frequency. This first stream of data includes digital markers (i.e., the timing-pulse) identifying the temporal location of the physiological QRS complex within the ECG data to the ultrasound echo imaging system. A real-time RF transmission can achieve the low data-latency required by the Association for the Advancement of Medical Instrumentation (AAMI) of less than 35 milliseconds for medical echo imaging. The second stream of data is transmitted by standard wireless transmission technology (such as Bluetooth or Wi-Fi). This second stream of data includes complete and reliable diagnostic quality of data which can be stored in a tangible electronic medium or displayed on a screen for review by a medical professional. The separate real-time transmission of digital markers enables a receiving processor to re-synchronize the first set of data and the second set of data in the echo imaging process such that the ECG data can be displayed synchronously with the echo image(s) such that the ECG data is synchronously displayed with the echo images that depict the corresponding physical activity of the anatomy being imaged.

Embodiments of the present invention include a real-time digital QRS sync-pulse transition, and the near real-time, full fidelity, diagnostic quality ECG transmission stream that both contain the same relative QRS interval timing even though both data streams are received separately by the echo imaging/display system at different times. Using the relative QRS complex timing, and other digital information contained within the transmission streams, the near real-time ECG signal can be re-synchronized by a processor by the echo application. The real-time pulse signal can provide synchronization for the echo system image capture, and the near real-time ECG signal can be used to display a true ECG signal with the echo images in review, immediately after the image capture. In some embodiments, a software algorithm on the host medical system re-synchronizes the two data streams using the relative timing between events or the unique keys or counters in near real-time for human observation. Embodiments of the invention can provide the full fidelity data signal to be transmitted using available wireless transmission technology, while still retaining the deterministic, low latency, real-time notifications required to combine ECG stress test data with live medical imaging.

### BRIEF DESCRIPTION OF THE DRAWINGS

The embodiments of the present invention may be more completely understood in consideration of the following detailed description of various embodiments of the invention in connection with the accompanying drawings, in which:

FIG. 1 is a block diagram of an echo/ECG real-time synchronization system according to an embodiment of the present invention.

FIG. 2a is a block diagram of an echo/ECG real-time synchronization system according to an embodiment of the present invention.

FIG. 2b is a block diagram of ECG/QRS transmitter and receiver modules according to an embodiment of the invention.

FIG. 3 is a flow chart depicting the collection, transmission, and re-synchronization of multiple medical data signals according to an embodiment of the present invention.

FIG. 4 is a block diagram of a wireless echo/ECG real-time synchronization system according to an embodiment of the present invention.

FIG. 5 is a block diagram of a wired echo and wireless ECG real-time synchronization system according to an embodiment of the present invention.

While the present invention is amendable to various modifications and alternative forms, specifics thereof have been shown by way of example in the drawings and will be described in detail. It should be understood, however, that the intention is not to limit the present invention to the particular embodiments described. On the contrary, the intention is to cover all modifications, equivalents, and alternatives falling within the spirit and scope of the present invention.

### DETAILED DESCRIPTION OF THE DRAWINGS

By way of example, the following discussion will assume interfacing an ECG monitor with an ultrasound echo imaging device, although, any imaging system can be used.

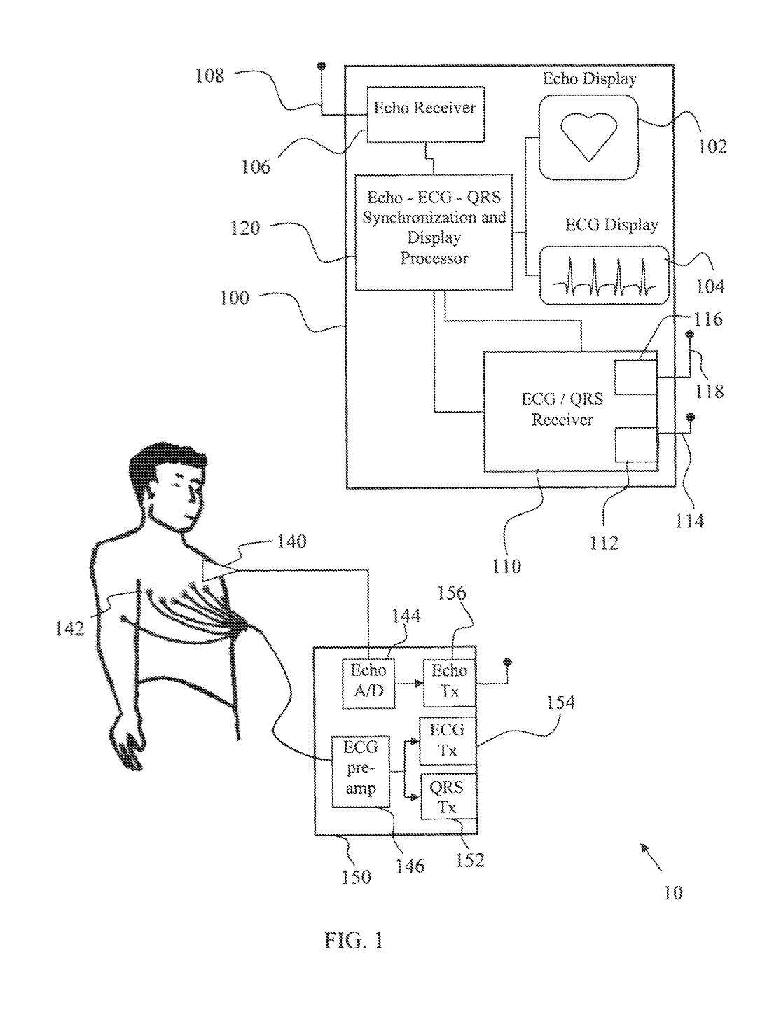

Referring to FIG. 1, an embodiment is shown of an echo/ECG real-time synchronization system 10. In this embodiment, a display unit 100 can present acquired patient data with an echo image display 102 and an ECG display 104. The display unit 100 can include a non-volatile computer-readable storage medium to record and store image and ECG data for later review, as well as a network connection to transmit or receive image or ECG data to or from a remote server. The image presented in the echo image display 102 is received wirelessly by an echo receiver 106 and corresponding antenna 108. The image presented in the ECG display 104 is received wirelessly by a ECG/QRS receiver 110 that includes a QRS receiver 112 and corresponding QRS antenna 114, and a ECG receiver 116 and corresponding ECG antenna 118. The three signals (echo, QRS, and ECG) are synchronized by a processor 120 that provides the image data to the echo image display 102 and the ECG display 104.

An ultrasound transducer 140 and a set of ECG sensors 142 that are proximate to the patient can acquire patient data. The ultrasound transducer 140 provides an ultrasound transducer signal to an echo A/D converter 144. The ECG sensors provide ECG data to a pre-amp 146. The A/D converter 144 and ECG data to a pre-amp 146 can be contained in separate apparatus, or be combined into a single patient data acquisition module 150. Patient data acquisition module 150 can include a QRS transmitter 152 that transmits QRS data derived from the ECG data obtained by sensors 142 over a low-latency channel to the QRS radio receiver 112, an ECG transmitter 154 that transmits patient ECG data acquired from the ECG sensors 142 over a high-bandwidth channel to the ECG radio receiver 116, and an echo transmitter 156 that transmits the echo image data acquired from the ultrasound transducer 140 to the echo receiver 106. The QRS data and the echo image data are both transferred over a low-latency protocol such that the transmission of both types of data are in sync with each other.

The QRS transmitter 152 can generate a RF radio transmission to transmit a digital marker identifying the temporal location of the physiological QRS complex to the display unit 100 to allow for real time synchronization of the echo imaging process, with the patient physiological events (data latencies meeting the AAMI requirements of no more than thirty-five milliseconds). Such data delivery with a latency of thirty-five milliseconds or less is considered to be of “low latency” for purposes of this application. The ECG transmitter 154 can include standardized wireless transmission technologies that can transmit the full diagnostic quality data obtained from the ECG sensors 142 using known error correction and retry techniques to guarantee reliable delivery of the ECG data in near real time (data latencies up to several seconds). Such data delivery with a latency of more than thirty-five milliseconds is considered to be of “high latency” for purposes of this application.

The real-time QRS signal, which can be a digital sync pulse transition, and the near real-time, full fidelity, diagnostic quality ECG transmission stream both contain the same relative QRS interval timing even though both data streams are received by the ECG receiver 116 and the QRS receiver 112 at different times. Using the relative QRS complex timing, and other digital information contained within the transmission streams, the near real-time ECG signal can be re-synchronized with the echo image data by the processor 120 by deriving the QRS complex timing information from the full fidelity ECG transition and calculating the offset time between the receipt of the QRS signal received at the same time the image data was received and the time the ECG data was received. The real-time pulse signal will provide synchronization for the echo system image capture, and the near real-time high-bandwidth ECG signal can be used to display a true and accurate ECG signal that is synchronized with the echo images. This provides for the review, immediately after the capture, of both the full resolution ECG signal and the echo images acquired by the transducer 140 when both the ECG and the echo images are transferred over separate wireless links.

Echo imaging systems generally require a data latency or phase shift of 35 milliseconds or less from the physiological event to input of the electronic signal for imaging synchronization. For purposes of this application, such data latencies of 35 milliseconds or less are considered to be of “low latency”. Because the data latency needs to be deterministic or constant (isochronous), and standard off-the-shelf (OTS) wireless transmission protocols typically have a non-deterministic data latency of several hundred milliseconds or greater, the retry and error detection and correction logic built into OTS protocols makes the data latency variable and non-deterministic. Data latencies higher than 35 milliseconds, like these, are considered to be of “high latency” for purposes of this application, and accordingly the wireless data latencies in standard off-the-shelf wireless transmission protocols are of high latency. For these reasons, previously known Stress and echo systems were generally integrated using a hardwired, low-level analog data cable, which forgoes the advantages of a wireless patient connection.

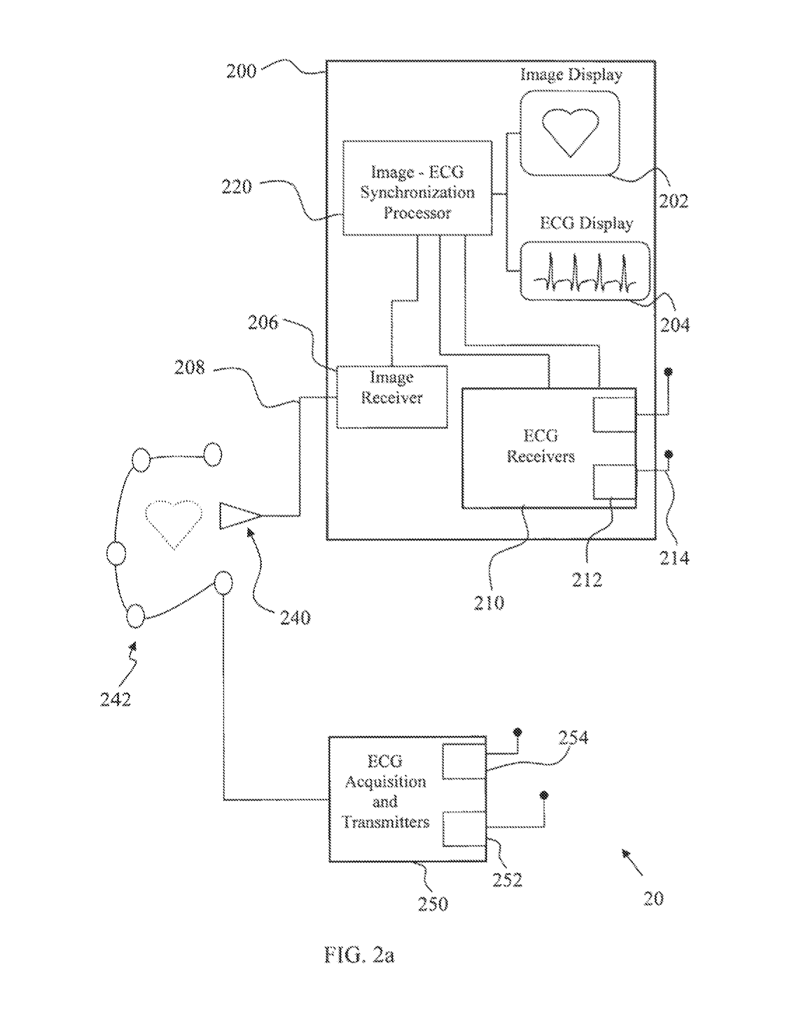

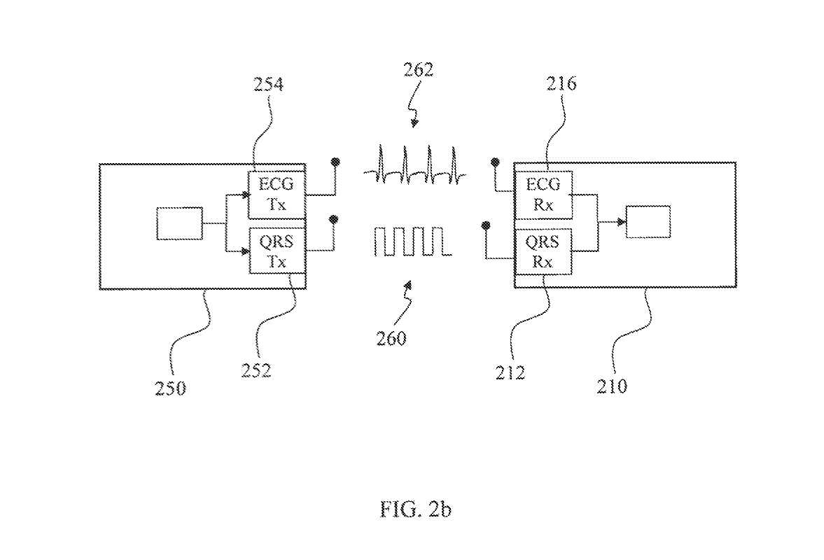

Referring to FIGS. 2a and 2b, an embodiment of the present invention depicting a echo/ECG real-time synchronization system 20 and a diagram of ECG/QRS transmitter and receiver modules is shown. The embodiment allows the use of a wireless patient connection via a patient module data-acquisition device 250 to medical test equipment 200 that requires very low data latency for notification of physiological events. As depicted in FIG. 2a the medical test equipment 200 can include an imaging instrument 240 that is hardwired to the medical test equipment 200. Current wireless patient data acquisition systems are unable to provide the low data latency in combination with a wireless solution and also maintain reliable data delivery.

The patient module data-acquisition device 250 includes a for high-bandwidth full-resolution ECG data transmitter 254 and low-latency QRS sync signal transmitter 252 to provide both ultra-reliable high bandwidth data transmission in combination with low-latency deterministic delivery that allows for the simultaneous display of ECG and echo data.

The patient module data-acquisition device 250 can provide both reliable wireless data transmission and low data latency by breaking the data signals acquired from a patient into multiple parts that can be resynchronized by Image-ECG processor 220. As depicted in FIG. 2b, a QRS data channel 260 can consist of limited digital event-markers to alert the medical test equipment 200 of physiological events important to the function and performance of medical test equipment 200. Key physiological events are marked with a RF radio transmission of a key or code along with an optionally unique event identifier (key or counter) to preserve the temporal relationship to the true physiological event. This RF transmission can occur with known, deterministic, data latency but need not have ultra-high reliability. The QRS data channel 260 can contain a digital representation of the relative or absolute timing of the physiological events.

A ECG data channel 262 can consist of standard off-the-shelf wireless protocol technologies (for example, but not limited to, Wi-Fi or Bluetooth technologies) and can transmit acquired patient information in a high-resolution format. The ECG data channel 262 (which generally uses high bandwidth transmissions) would not need to meet the low data latency requirements required of the QRS data channel 260 (which generally uses low bandwidth transmissions), but would be delivered using reliable, error-correcting data delivery technologies and protocols. The ECG data channel 262 can also contain (or allow the derivation of), the physiological event markers transmitted in the low latency data channel such that both channels can be synchronized at the receiving system. Alternatively, both the ECG data channel 262 and the QRS data channel 260 can contain an embedded time stamp or other real-time sequence indicator that would allow for the calculation of any temporal offset of the two data channels at a data receiving module 210 that includes the QRS receiver 212 and ECG receiver 216.

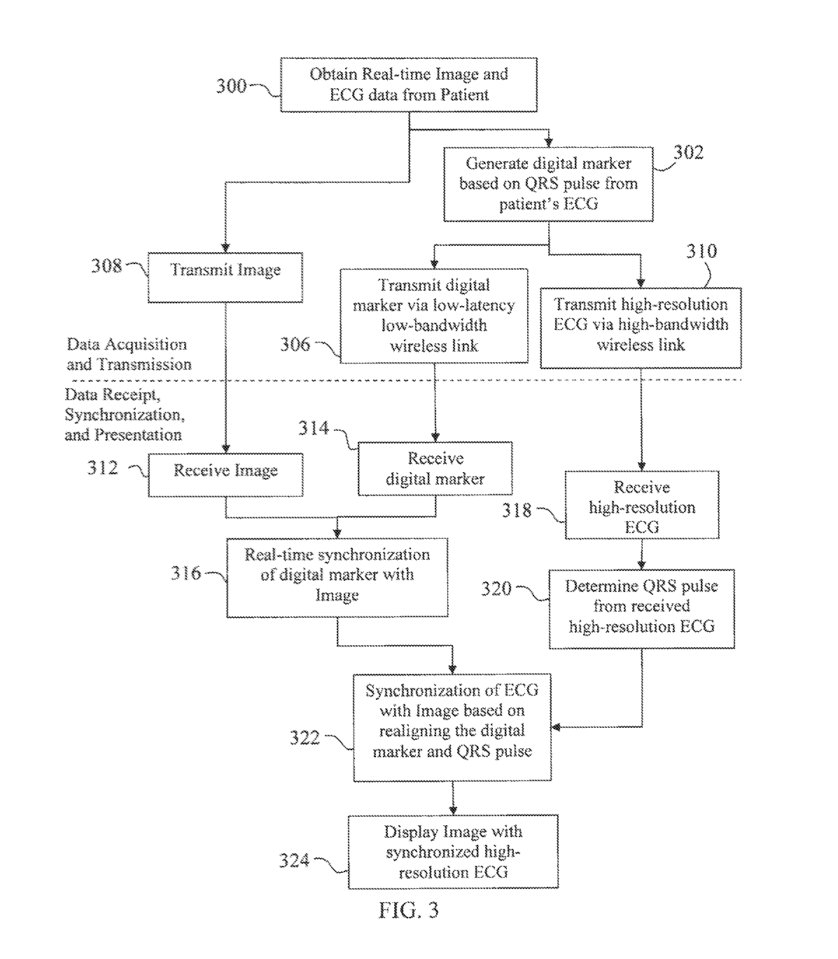

Referring to FIG. 3, a software algorithm in a host medical system can synchronize two separate data streams using the relative timing between events and/or the unique keys or counters in near real-time for human observation. This can provide the full fidelity data signal to be transmitted, while still retaining the deterministic, low latency real-time notifications required to synchronize the wirelessly transmitted data with real-time imaging data.

Real-time image and ECG data are both simultaneously acquired from the patient (step 300). This acquisition provides two separate data streams, the image data and the ECG data. From the acquired ECG data a digital marker is generated based on the QRS pulse embedded in the ECG data, or another appropriate event that can serve as a periodic time stamp that can be derived from the ECG data (step 302). The generated digital marker is transmitted over a low-latency, and optionally low-bandwidth, first wireless link (step 308) at the same time the image data is being transmitted to an image display station (step 306). The high-resolution ECG data is transmitted over a high-bandwidth second wireless link (step 310), ideally at the same time as the image and digital marker data, however the high-resolution ECG data does not need to be in perfect synchronization with the other two signals. The high-resolution ECG data contains sufficient information to independently generate the digital marker.

At the image display station the image data is received (step 312) at the same time as the digital marker data is received (step 314) such that the image data and the digital marker data are synchronized in real-time (step 316). In this example, real-time synchronization can be defined as having an offset of less than thirty-five milliseconds. The high-resolution ECG data is also received at the image display station (step 318), however the ECG data can lag behind the image and digital marker data due to the use of data-correction or other latency introducing processing that can be necessary to ensure that the high-resolution ECG data is fully and accurately communicated. While in an ideal situation the lag time between the digital marker and the ECG data would be zero, the lag can be greater than thirty-five milliseconds (i.e. greater than low latency transmissions) but preferably less than one or two seconds (i.e. within the definition of high data latency).

As the high-resolution ECG data is received a second digital marker is again generated from the ECG data that corresponds to the first digital marker (step 320). One or more buffers can be utilized to accommodate the offset in time between the receipt of the image data and the high-resolution ECG data. By aligning the real-time digital marker that was received with the image data with the second digital marker obtained from the received ECG data (step 322) both the image data and the high-resolution ECG data can be presented simultaneously (step 324) or stored for later review.

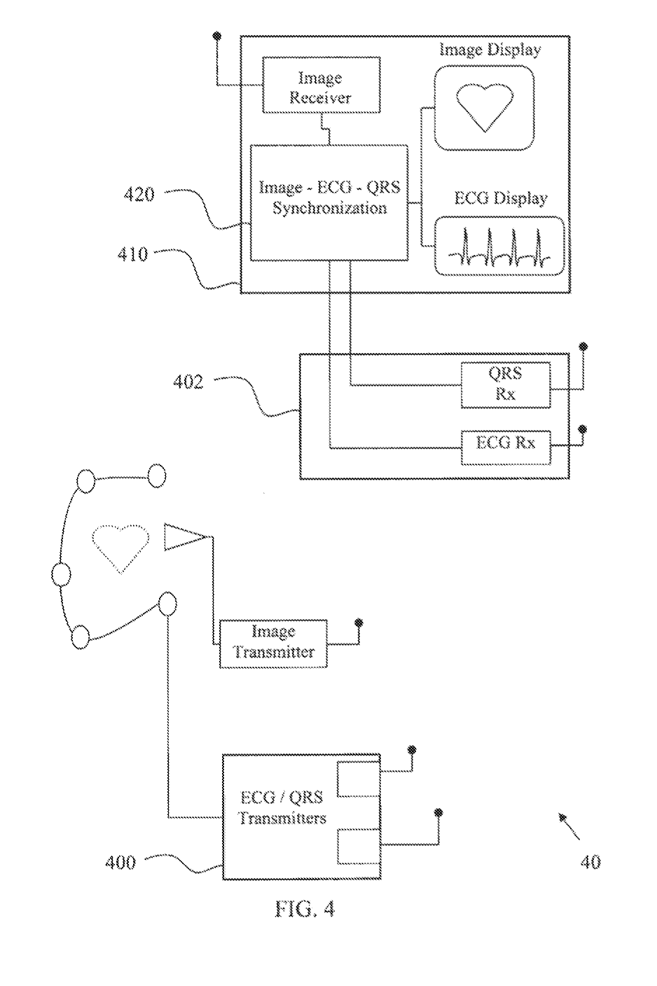

Referring to FIG. 4, a further embodiment having a wireless echo/ECG real-time synchronization system 40 is shown. In this embodiment, a stress-test application using a wireless patient data acquisition transmitter 400 and receiver 402 pair can be connected to any other system/device requiring an ECG signal and QRS trigger (the digital marker) allowing Stress Testing modality solutions with a wireless preamp to integrate with a third-party image display device 410.

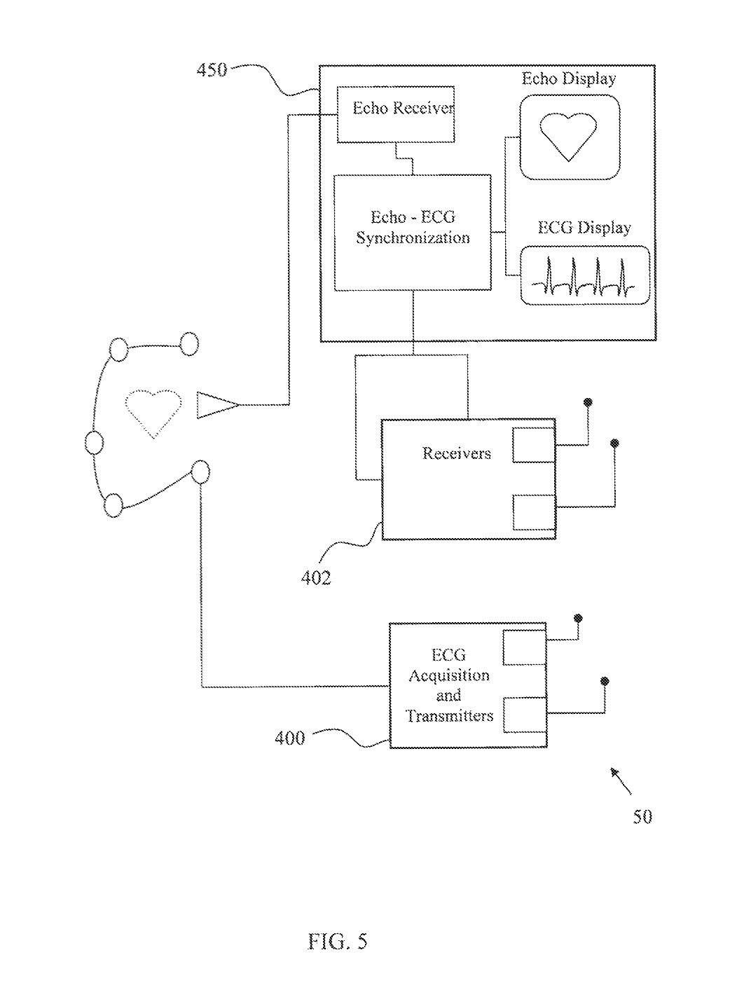

Referring to FIG. 5, a further embodiment having a wired echo and wireless ECG real-time synchronization system is shown. In this embodiment, a stress-test application using a wireless patient data-acquisition transmitter 400 and receiver 402 pair can be connected to any other system/device requiring an ECG signal and QRS trigger allowing Stress Testing modality solutions with a wireless preamp to integrate with third-party wired image device 450.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

What is claimed:

1. A multi-channel wireless system for medical electrocardiogram (ECG) data and echocardiogram (echo) image data comprising:
a medical data acquisition apparatus configured to acquire ECG data and echo image data from a plurality of sensors;
a first radio transmitter, configured to utilize a low-latency data protocol, operably coupled to the medical data acquisition apparatus and configured to transmit a first portion of the ECG data simultaneously with a separate transmission of the echo image data;
a second radio transmitter having a high-bandwidth data protocol operably coupled to the medical data acquisition apparatus, and configured to transmit a high-resolution data stream of the ECG data received from the medical data acquisition apparatus, the high-resolution data stream including the first portion of the ECG data;
a first radio receiver configured to receive the first portion of ECG data via the low-latency data protocol from the first radio transmitter;
a second radio receiver configured to receive the high-resolution data stream of the ECG data via the high-bandwidth data protocol; and
a processor operably coupled to the first radio receiver and the second radio receiver, wherein the processor is configured to synchronize the high-resolution data stream of ECG data with the echo image data based on the correspondence of the first portion of the ECG data with a temporal receipt of the echo image data.

2. The system of claim 1, the medical data acquisition apparatus including a processor configured to derive the first portion of the ECG data from the high-resolution data stream of ECG data received from the medical data acquisition apparatus.

3. The system of claim 1, further comprising a display to present the echo image data and the high-resolution data stream of ECG data.

4. The system of claim 1, wherein the echo image data is received via a wireless communication link.

5. The system of claim 1, wherein the echo image data is received via a wired communication link.

6. The system of claim 1, wherein the high-resolution data stream has a data latency of more than 35 milliseconds.

7. The system of claim 1, wherein the low-latency data protocol has a data latency of 35 milliseconds or less.

8. The system of claim 1, wherein the plurality of sensors includes a five-lead electrocardiogram sensor array.

9. The system of claim 1, wherein the plurality of sensors includes a twelve-lead electrocardiogram sensor array.

10. A method of synchronizing echocardiogram (echo) medical image(s) with high-resolution electrocardiogram (ECG) medical data acquired from a patient, the method comprising:
obtaining an echo image;
obtaining a ECG data signal simultaneously with the obtaining of the echo image;
generating a temporal marker based on a marker in the ECG data signal;
delivering the echo image to a receiver;
transmitting the temporal marker on a low-latency wireless link;
coordinating the receipt of the temporal marker and the echo image at the receiver;
transmitting the ECG data signal on a high-reliability high-bandwidth wireless communication protocol; and
synchronizing the ECG data signal with the echo image based on the association of the temporal marker with the ECG data signal and the coordinated receipt of the temporal marker with the echo image.

11. The method of claim 10, wherein the low latency wireless link has a data latency of 35 milliseconds or less.

12. The method of claim 10, wherein the high-reliability high-bandwidth wireless communication protocol has a data latency of more than 35 milliseconds.

13. The system of claim 10, wherein the echo image is delivered via a wireless communication link.

14. The system of claim 10, wherein the echo image is delivered via a wired communication link.

15. A tangible computer-readable medium, storing instructions executed by a computer system to implement a method for receiving and synchronizing echocardiogram (echo) medical image(s) and high-resolution electrocardiogram (ECG) medical data, the instructions comprising:
instructions for generating a temporal marker based on a marker embedded in a ECG data signal obtained simultaneously with the generation of a real-time echo image at a first location;
instructions for transmitting the temporal marker on a low-latency wireless link to a receiver in conjunction with the transmission of the echo image;
instructions for transmitting the ECG data signal on a high-reliability high-bandwidth wireless communication protocol;
instructions for coordinating the receipt of the temporal marker and the echo image at a receiver located at a second location; and
instructions for synchronizing the ECG data signal with the echo image based on the association of the temporal marker with the ECG data signal and the coordinated receipt of the temporal marker with the echo image.

16. The tangible computer-readable medium of claim 15, wherein the low latency wireless link has a data latency of 35 milliseconds or less.

17. The tangible computer-readable medium of claim 15, wherein the temporal marker is a QRS pulse embedded in the ECG data signal.

18. The tangible computer-readable medium of claim 15, wherein the high-reliability high-bandwidth wireless communication protocol has a data latency of more than 35 milliseconds.
